(19) United States
(12) Patent Application Publication
(10) Pub. No.: US 2026/0174544 A1
(43) Pub. Date: Jun. 25, 2026

(54) Suture Anchors and Methods of Tissue Repairs

(71) Applicant: Arthrex, Inc., Naples, FL (US)

(72) Inventors: Thomas DOONEY, JR., Naples, FL (US); Matthew R. HERRINGTON, Naples, FL (US); John A. SODEIKA, Naples, FL (US); Jason A. VALENTIN, Fort Myers, FL (US); Loren D. CROOK, Fort Myers, FL (US)

(73) Assignee: Arthrex, Inc., Naples, FL (US)

(21) Appl. No.: 19/426,530

(22) Filed: Dec. 19, 2025

Related U.S. Application Data
(60) Provisional application No. 63/738,583, filed on Dec. 24, 2024.

(51) Int. Cl. A61F 2/08; A61B 17/04

(52) U.S. Cl. CPC A61F 2/0811; A61B 17/0401; A61B 17/0466; A61B 2017/0414; A61B 2017/0445; A61B 2017/0464; A61F 2002/0823; A61F 2002/0852; A61F 2002/0864

(57) ABSTRACT

Surgical constructs, assemblies and methods for surgical repairs are disclosed. A soft suture anchor with a fixed suture limb or limbs is secured within tissue. The suture limb may be passed through soft tissue and within the tissue at various locations and in a sequential manner to form a stitching pattern over the soft tissue. The suture limb may be fixated laterally within the tissue with an independent fixation device.

### CROSS-REFERENCE TO RELATED APPLICATIONS

The application claims priority to U.S. Provisional Patent Application No. 63/738,583 filed Dec. 24, 2024, the disclosure of which is hereby incorporated by reference in its entirety herein.

### BACKGROUND

The disclosure relates to the field of surgery and, more specifically, to anchor constructs, assemblies and associated methods of tissue repairs.

### SUMMARY

Surgical systems, assemblies, and methods for fixation of soft tissue are disclosed.

A surgical construct may create a reinforced, stitched repair. A surgical construct may be made completely of suture to achieve increased fixation of soft tissue to bone. A suture anchor with one or more fixed limbs may be secured into bone and employed to form a stitch or sewing pattern over the soft tissue attached to bone.

Methods of tissue repairs are also disclosed. In some implementations, a plurality of surgical constructs may be secured into a first tissue. At least one of the surgical constructs may include a fixed suture limb which is passed over a second tissue to be positioned to the first tissue in a serial manner, to form a plurality of stitches. The fixed suture limb may be secured into the first tissue by being passed through the other surgical constructs.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a surgical construct.

FIG. 2 illustrates another surgical construct.

FIG. 3 illustrates another surgical construct.

FIG. 4 illustrates another surgical construct.

FIG. 5 illustrates a surgical repair with surgical constructs.

FIG. 6 illustrates a surgical repair subsequent to that of FIG. 5.

FIG. 7 illustrates a surgical repair subsequent to that of FIG. 6.

FIG. 8 illustrates a surgical repair subsequent to that of FIG. 7.

FIG. 9 illustrates a surgical repair subsequent to that of FIG. 8.

FIG. 10 illustrates a surgical repair subsequent to that of FIG. 9.

FIG. 11 illustrates a surgical repair subsequent to that of FIG. 10.

FIG. 12 illustrates another surgical repair with surgical constructs.

FIG. 13 illustrates another surgical repair with surgical constructs.

### DETAILED DESCRIPTION

A surgical construct may create a stitched repair. The repair may be tensionable, reinforced, self-locking.

A surgical anchor may be made completely of suture to achieve fixation of first tissue (soft tissue, graft, tendon, etc.) to a second tissue (bone). The surgical anchor may be provided with a soft body and one or more fixed single tails (single limbs) that remain outside the soft tissue and are employed to stitch or sew the first tissue to the second tissue. The one or more fixed single tails may form a plurality of suture passes and stitching patterns to secure soft tissue to bone. The one or more fixed single limbs may be secured at various locations within the bone. The stitching pattern may be linear or non-linear, or combinations thereof. The stitching pattern may have any geometrical shape such as triangular, rectangular, etc.

A surgical assembly may include a plurality of fixation devices and at least one flexible coupler that may be securely attached to a first tissue. The at least one flexible coupler may be passed serially through a second tissue (to be attached to the first tissue) at a plurality of locations within the first tissue to form a plurality of stitching passes. The at least one flexible coupler may be serially fixated within the first tissue at various locations (corresponding to the stitching passes) with a plurality of fixation devices. The at least one flexible coupler may be laterally fixated within the first tissue with at least one independent fixation device.

Methods of tissue repairs are also disclosed. A plurality of surgical constructs may be secured into a first tissue. At least one of the surgical constructs includes a fixed suture limb which is passed over a second tissue to be attached to the first tissue to connect the other of the surgical constructs in a serial manner, to form a plurality of stitches over the second tissue. The fixed suture limb may be secured laterally into the first tissue with an independent fixation device.

In some implementations, a surgical construct provides tissue fixation by providing an all-suture soft anchor provided with a single limb that may be employed to knotlessly fixate soft tissue to bone. The single limb may be passed through soft tissue to form a plurality of stitches or suture passes at various locations. The single limb may be secured within hard tissue with additional soft anchors corresponding to the various locations and suture passes.

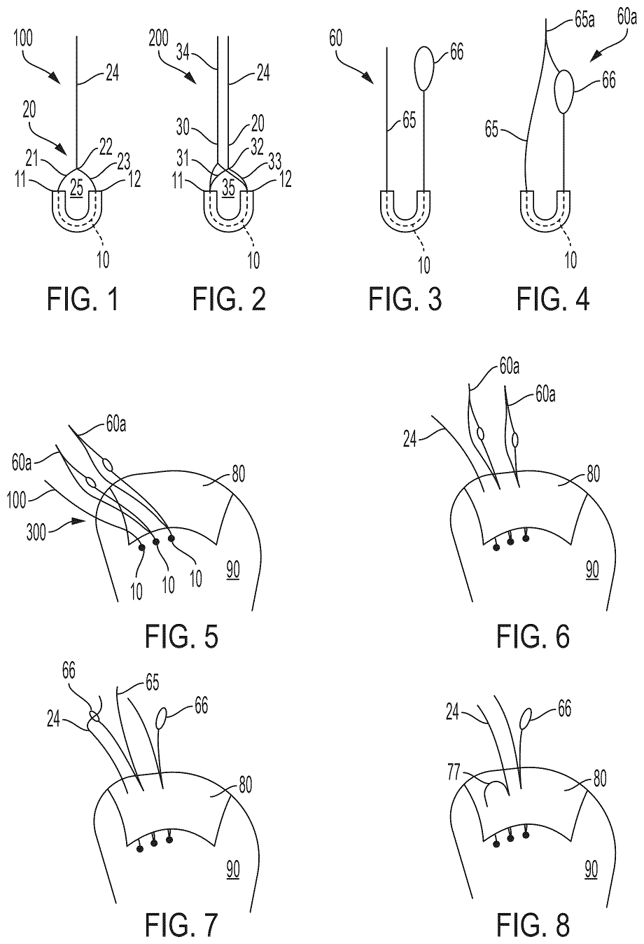

Referring now to the drawings, where like elements are designated by like reference numerals, FIGS. 1 and 2 illustrate surgical constructs 100, 200. FIGS. 3 and 4 illustrate shuttling construct 60, 60a. FIGS. 5-11 illustrate exemplary steps of surgical repair 101 with surgical construct 100. FIGS. 12 and 13 illustrate surgical repairs 201, 301 with surgical constructs 100, 200.

Surgical construct 100, 200 may be an implantable device; a suture anchor; an implant; a surgical assembly; a surgical system; a suture tape anchor; a tensionable, knotless construct; a tensionable, knotted construct; a single limb soft anchor; a single limb FiberTak® construct; a SutureTape FiberTak® construct; or a fixation device.

Construct 100, 200 is supported by soft or hard tissue and produces at least one fixed repair limb for a custom tissue to tissue repair. The device may consist of an anchor body 10 and at least a flexible coupler 20, 30 with a fixed limb 24, 34 (fixed suture limb 24, 34). As detailed below, the fixed repair limb may be used for additional steps, to pass through soft tissue at different locations to form suture passes or stitches, and to be secured into hard tissue at different locations with additional fixation devices. The additional steps may be sequential steps to form various sewing and/or stitching patterns for attaching the soft tissue to the hard tissue. Fixation of the fixed suture limb or limbs within the hard tissue may be conducted with knotless or knotted fixation devices, for example, knotless suture anchors.

FIG. 1 illustrates exemplary surgical construct 100 with anchor body 10 (fixation device 10) that may be in the form of a soft suture anchor (soft suture sheath, soft suture anchor, all-suture soft knotless anchor, implant) provided with a soft anchor sleeve 10 (sheath or tubular member 10) with two open ends 11, 12. Flexible coupler 20 extends through soft anchor sleeve 10 and may exit each of the two open ends 11, 12. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

Flexible coupler 20 may be formed of one single continuous coupler in form of suture, either round and/or flat suture, for example, a suture strand or suture tape. Flexible coupler 20 may be provided with two terminal ends, a first end 21 and a second end 23. Flexible coupler 20 may be a fixed length of suture and/or tape, such as suture tape. As detailed below, flexible coupler 20 may be attached to one or more independent fixation devices (such as fixation device 60, 60a of FIGS. 3 and 4), to form a plurality of suture passes as part of a sewing pattern.

Flexible coupler 20 may extend through the anchor sleeve 10 in similar or different directions and/or orientations and/or locations. Flexible coupler 20 may extend through the whole length of the sleeve, e.g., enters one of the two open ends 11, 12 and exits the other of the two open ends 11, 12. In other implementation, flexible coupler 20 may extend through only a portion of a length of the sleeve 10, e.g., may enter the sleeve at a location separate from one of the two open ends 11, 12 and may exit at a location separate from other of the two open ends. In yet other implementations, flexible coupler 20 may enter the sleeve 10 at an open end and may exit the sleeve at a location spaced apart from the other open end.

In some implementations and as shown in FIG. 1, surgical construct 100 is formed by passing flexible coupler 20 through the anchor sheath 10 and with ends 21, 23 brought together at location 22 to form fixed suture limb 24 and flexible loop 25 having a fixed, non-adjustable length. Location 22 may include a knot (for example, a small static knot) or a similar structure (such as a splice, bonded area, glued area, etc.) that brings the two ends 21, 23 together resulting in a single, fixed repair limb 24.

In some implementations and as shown in FIG. 2, surgical construct 200 may include two exemplary fixed suture limbs 24, 34. Construct 200 may be formed by passing one of two terminal ends 21, 23 (for example, first end 21) of first flexible coupler 20 through soft anchor 10 in a first direction and from a first end 11 to a second end 12, and then bringing together the two ends 21, 23 to form a first fixed suture limb 24. The steps may be repeated for a second flexible coupler 30 to form a second fixed suture limb 34. One of terminal ends 31, 33 of flexible coupler 30 may be passed through soft anchor 10 in a second direction. The second direction may be similar to, or different from, the first direction. The second direction may be opposite the first direction. Terminal ends 31, 33 may be brought together at location 32 to form second loop 35 with a fixed perimeter and fixed length, and a second fixed suture limb 34.

Flexible coupler 20, 30 may be passed through at least a portion of the body of sheath 10. The passes may be formed with suture passers and/or shuttle/pull devices and/or suture passing instruments such as needles, FiberLink™ loops, nitinol loops, or any suture passing device that includes an eyelet/loop for passing the flexible coupler 20, 30 through tubular member or sheath 10. One or more of flexible couplers 20, 30 may be fixedly attached to the sheath 10. In some implementations, both flexible couplers 20, 30 may be non-slidingly and fixedly attached to sheath 10.

Constructs 100, 200 may be individually assembled on inserters or multiple anchors 100, 200 may be assembled into one delivery device.

FIGS. 3 and 4 illustrate fixation device 60, 60a in the form of a suture anchor 60, 60a with sheath 10 (body anchor 10) and a shuttle stitch 65 with shuttling loop 66. FIG. 4 shows suture anchor 60a with shuttle stitch 65 that is combined to one tail 65a for easy passing. Single tail 65a may be cut and be open (FIG. 3) to allow for shuttling the suture limbs 24, 34, as detailed below. Plurality of anchors 100, 200, 60 (60a) form assembly 300 (as detailed below with reference to FIG. 5).

Reference is now made to FIGS. 5-11 which illustrate exemplary steps of surgical repair 101 with surgical construct 100. FIGS. 12 and 13 illustrate surgical repairs 201, 301 with surgical constructs 100, 200. Surgical repair 101, 201, 301 may be rotator cuff repair.

FIG. 5: Anchors 100, 60a may be positioned under first tissue 80 and within second tissue 90. First tissue 80 may be soft tissue, tendon, ligament, graft, etc. Second tissue 90 may be hard tissue such as bone. Surgical construct 100 attaches first tissue 80 to second tissue 90 with aid of two exemplary fixation devices 60a. Construct 100 and fixation devices 60a form surgical assembly 300 which may be part of various surgical kits. Anchors 100, 60a may be inserted into bone 90 or directly through tissue 80 into bone 90 (in a transtendon approach). In this implementation, the sutures do not need to be passed through tissue 80.

FIG. 6: Sutures 24, 65a from anchors 100, 60a are passed through first tissue 80.

FIG. 7: The shuttle stitch 65 is cut to free up the ends. The repair suture 24 is fed into the shuttle 66 of anchor 60.

FIG. 8: The shuttle stitch 65 is pulled to shuttle the repair suture 24 through the anchor sheath 10 of anchor 60. Flexible limb 24 passes through first tissue 80 and is fixated with fixation device 60 into second tissue 90. Fixation device 60 may be an exemplary knotless anchor. The repair stitch 65 may be tensioned to reduce and fixate against the tissue. In some implementations, anchor 60 may be just a pulley, so the repair stitch may not be technically “fixated.” FIG. 8 illustrates suture pass 77 or stitch 77 formed by repair suture 24 over and through soft tissue 80 to be attached to bone 90.

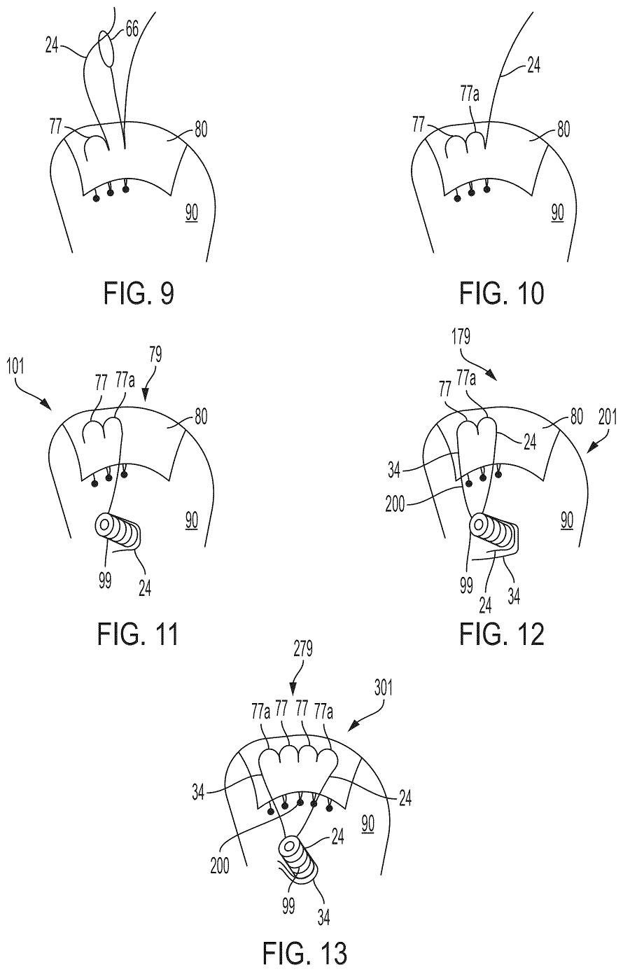

FIGS. 9 and 10: The repair suture 24 is fed into the shuttle 66 of another, second fixation device 60. The shuttle stitch 65 is pulled to shuttle the repair suture 24 through the anchor sheath 10 of second anchor 60 and form another stitch 77a. Flexible limb 24 passes through the first tissue 80 at a different location and is fixated with second fixation device 60 into second tissue 90. Fixation device 60 may be another knotless anchor. The steps may be repeated as necessary with multiple anchors 60 (60a).

FIG. 11 illustrates exemplary generic repair 101 which may be a rotator cuff repair. Suture limb 24 (repair stitch 24) is tensioned to reduce the tissue 80 to bone 90. The repair stitch 24 may be fixated with another, independent fixation device 99 laterally, as part of repair 101. Fixation device 99 may be a knotless anchor such as a SwiveLock® anchor 99, so that repair 101 is a knotless repair. One or more independent fixation devices 99 may be employed. Repair 101 illustrates two exemplary passes or stitches 77, 77a as part of stitching pattern 79; however, the disclosure contemplates any number of passes/stitches formed with any number of anchors 60 (60a) and shuttling stitches 65 with loops 66, as necessary for the final repair.

FIG. 12 illustrates another exemplary repair 201 which may be a rotator cuff repair. Repair 201 is about similar to repair 101 detailed above in that repair 201 also provides a stitching pattern 179 and is conducted with two fixation devices 60 (60a). However, repair 201 employs construct 200 (FIG. 2) which is loaded with multiple suture limbs 24, 34 (repair stitches 24, 34). Repair stitch 24 forms stitching pattern 179 with exemplary fixation devices 60 (60a). After forming stitching pattern 179, repair limb 24 may be fixated laterally together with repair limb 34 with one or more fixation devices 99 as part of repair 201. Fixation device 99 may be a knotless anchor such as a SwiveLock® anchor 99, so that repair 201 is a knotless repair.

FIG. 13 illustrates yet another exemplary repair 301 which may be a rotator cuff repair. Repair 301 is about similar to repairs 101, 201 detailed above in that repair 301 also provides a stitching pattern 279 and employs construct 200 (FIG. 2) that is loaded with multiple suture limbs 24, 34 (repair stitches 24, 34). However, repair 301 is conducted with four fixation devices 60 (60a) to secure the two repair stitches 24, 34 to bone 90. After forming stitching pattern 279, repair limbs 24, 34 may be fixated laterally and together with one or more fixation devices 99, as part of repair 301. Fixation device 99 may be a knotless anchor such as a SwiveLock® anchor 99, so that repair 301 is a knotless repair.

Repair 301 illustrates four exemplary passes or stitches 77, 77a as part of stitching pattern 279; however, the disclosure contemplates any number of passes/stitches and any number of fixation devices 60 (60a), as necessary for the final repair 301.

In some implementations, the one or more independent fixation devices 99 may be any anchors, for example, any of knotted anchors, knotless anchors, or all-suture anchors, or any devices that confer secure attachment and fixation of first tissue to a second tissue. Fixation device 99 may be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

The constructs, systems, and assemblies of the present disclosure may be employed in various soft tissue repairs and fixations, for example, fixation of soft tissue to bone.

Serial, in-line, suture-based, arthroscopic tissue repair devices and constructs of the present disclosure may be employed for any tissue positioning and/or tissue adjustment applications, for example, in fixation of soft tissue to bone. The suture implant/assembly and the sewing methods of the present disclosure may be utilized in surgical procedures such as rotator cuff repair, Achilles tendon repair, patellar tendon repair, ACL/PCL reconstruction, hip and shoulder reconstruction procedures, AC joint reconstruction, syndesmosis reconstruction, quad/patellar tendon rupture repair, hallux-valgus repair, proximal and/or distal biceps tendon repair, humerus and radius repair, and any other tendon repair to bone, among many others, all conducted in a knotless manner.

A surgical construct 100, 200 may be a fixation device with an anchor body 10 (sheath 10 or tubular member 10) and a flexible coupler 20, 30 attached to body 10. Flexible coupler 20, 30 may include a fixed suture limb 24, 34. Anchor body 10 is secured within hard tissue 90. Fixed suture limb 24, 34 may be passed through soft tissue 80 at various locations to form a plurality of suture passes 77, 77a or stitches 77, 77a as part of sewing or stitching pattern 79, 179, 279. Suture limb 24, 34 may form a plurality of serially connected suture passes 77, 77a or stitches 77, 77a as part of stitch pattern 79, 179, 279. Passing of suture limb 24, 34 through soft tissue 80 may be conducted with at least one suture anchor 60 (60a) with a shuttling stitch 65 and eyelet/loop 66. Suture limb 24, 34 may be secured laterally within bone 90 with an independent fixation device 99. A plurality of constructs 100, 200 may be secured within bone 90 and may form various stitching patterns having various configurations and geometries. The soft tissue 80 may be rotator cuff.

A surgical assembly 300 may include a plurality of fixation devices 100, 200, 60 (60a) and at least one flexible coupler 24, 34 that is securely attached to bone 90 and passed serially through soft tissue 80 at a plurality of locations. The at least one flexible coupler 24, 34 may be serially fixated within bone 90 at various locations (corresponding to the stitching passes 77, 77a and anchor bodies 10) with a plurality of fixation devices 60 (60a). The at least one flexible coupler 24, 34 may be laterally fixated within bone 90 with at least one fixation device 99.

Methods of soft tissue repair 101, 201, 301 which provide multiple points of fixation and/or stitching and/or sewing patterns 79, 179, 279 are also disclosed.

A method of tissue repair 101 may comprise inter alia the steps of: (i) securing a plurality of surgical constructs 100, 60 (60a) into a first tissue 90, wherein each of the surgical constructs 100, 60 (60a) includes a tubular sheath 10, wherein the surgical construct 100 includes a fixed suture limb 24 secured to the tubular sheath 10, and wherein surgical construct 60 includes a shuttling stitch 65 with an eyelet/loop 66, the shuttling stitch 65 slidingly passing through the tubular sheath 10; and (ii) passing the fixed suture limb 24 through and over a second tissue 80 to be positioned relative to the first tissue 90 in a serial manner to form a plurality of stitches 77, 77a and as part of stitching pattern 79, 179, 279. The method may further include (iii) laterally securing the fixed limb 24 to the first tissue 90 with an independent fixation device 99. Step (iii) may be conducted by securing the fixed limb 24 at a location lateral from that of the stitching pattern 79, 179, 279. Step (ii) may be conducted by shuttling the suture limb 24 through the second tissue 80 with one or more fixation devices 60 (60a) wherein each of the fixation devices 60 (60a) is secured into the first tissue 90; and by passing the suture limb 24 through the eyelet 66 and pulling the shuttling stitch 65 out of the tubular sheath 10 to pass the suture limb 24 through the tubular sheath 10 of each of the fixation devices 60 and out of the second tissue 80 to form a first stitch 77. The tissue repair may be rotator cuff.

A method of tissue repair 101, 201, 301 may comprise inter alia: (i) securing a plurality of fixation device 100, 200, 60 (60a) to a first tissue 90 (bone 90); (ii) passing at least one flexible coupler 24, 34 through a second tissue 80 (soft tissue 80) and through each sheath 10 of each of the plurality of fixation devices 60 (60a) to serially connect sheaths 10 of the plurality of fixation devices 60 (60a); and (iii) securing the flexible coupler 24, 34 to a lateral fixation device 99 into the first tissue 90. The method may further include tensioning the repair by pulling on flexible coupler 24, 34. Securing the plurality of fixation devices 60 (60a) in first tissue 90 may be conducted sequentially or in a serial manner. Passing the flexible coupler 24, 34 through the second tissue 80 and through the sheath 10 of each of the plurality of fixation devices 60 (60a) may be conducted sequentially or non-sequentially.

Suture limb 24, 34 may be any flexible coupler and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Suture limb 24, 34 may have cross-sections of various forms and geometries, including round, oval, rectangular, or flat, among others, or combination of such forms and geometries. In some implementations, suture limb 24, 34 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Suture limb 24, 34 may be braided or multi-filament suture such as FiberTape® suture tape (as disclosed in U.S. Pat. No. 7,892,256, the disclosure of which is incorporated in its entirety herewith). Suture limb 24, 34 may include elastic material. Suture limb 24, 34 may consist essentially of elastic suture.

Various regions or sections of suture limb 24, 34; tubular sheath 10; fixation devices 60 (60a); shuttling stitch 65 may be coated and/or provided in different colors for easy manipulation during the surgical procedures. These elements may be provided with tinted tracing strands, or otherwise contrast visually with the sheath 10 of the soft suture anchors 100, 200, 60 (60a) which remains a plain, solid color, or displays a different tracing pattern, for example. Easy identification of suture in situ is advantageous in surgical procedures, particularly during arthroscopic surgeries, endoscopic and laparoscopic procedures.

Any or all constructs 100, 200, 60 (60a) may be soft anchors such as an “all-suture” anchor. A soft anchor (soft suture anchor or all-suture soft knotless anchor) is provided with a soft anchor sleeve (sheath or tubular member) with two open ends, and at least one flexible shuttling strands extending through the soft anchor sleeve (sheath).

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A surgical repair, comprising:
a plurality of fixation devices secured within a first tissue; and
at least one flexible coupler attached to the plurality of fixation devices, the at least one flexible coupler forming in a knotless manner a plurality of suture passes over a second tissue to be attached to the first tissue.

2. The surgical repair of claim 1, wherein the plurality of suture passes are serially connected and extend between and connecting the plurality of fixation devices.

3. The surgical repair of claim 1, wherein the at least one flexible coupler is suture or suture tape.

4. The surgical repair of claim 1, wherein the at least one flexible coupler is fixedly attached to one of the plurality of fixation devices and is serially connected to each of the other of the plurality of fixation devices.

5. The surgical repair of claim 4, wherein each of the other of the plurality of fixation devices comprises a soft suture anchor with a tubular sheath or sleeve.

6. The surgical repair of claim 5, wherein each of the other of the plurality of fixation devices is provided with a shuttling stitch slidingly attached to the tubular sheath or sleeve, to allow passing of the at least one flexible coupler through the tubular sheath or sleeve of each of the other of the plurality of fixation devices.

7. The surgical repair of claim 5 comprising two flexible couplers fixedly attached to one of the plurality of fixation devices, wherein one of the two flexible couplers is serially connected to some of the plurality of fixation devices, and wherein other of the two flexible couplers is serially connected to other of the plurality of fixation devices.

8. The surgical repair of claim 7, wherein each of the some of the plurality of fixation devices is provided with a shuttling stitch slidingly attached to the tubular sheath or sleeve, to allow passing of the one of the two flexible couplers through the tubular sheath or sleeve of each of the some of the other of the plurality of fixation devices.

9. The surgical repair of claim 7, wherein each of the other of the plurality of fixation devices is provided with a shuttling stitch slidingly attached to the tubular sheath or sleeve, to allow passing of the other of the two flexible couplers through the tubular sheath or sleeve of each of the other of the plurality of fixation devices.

10. The surgical repair of claim 1, wherein the at least one flexible coupler is secured within the first tissue with a hard-body anchor at a location lateral to the plurality of suture passes.

11. The surgical repair of claim 1, wherein the repair is rotator cuff repair.

12. A surgical construct comprising a flexible coupler knotlessly connected to a plurality of soft anchors, wherein each of the plurality of soft anchors is secured within a first tissue, and wherein the flexible coupler is serially connected to each of the plurality of soft anchors and forms a stitching pattern over a second tissue, wherein the first tissue is different from the second tissue.

13. The surgical construct of claim 12, wherein the flexible coupler is fixedly connected to a fixation device secured within the first tissue.

14. The surgical construct of claim 13, wherein the fixation device is another anchor.

15. The surgical construct of claim 12, wherein the flexible coupler passes through a shuttling stitch and corresponding tubular sheath of each of the plurality of soft anchors.

16. The surgical construct of claim 12, wherein each of the plurality of soft anchors is an all-suture anchor, and the flexible coupler is round suture.

17. The surgical construct of claim 12, wherein the construct consists essentially of suture.

18. A surgical construct consisting essentially of a flexible coupler secured into a first tissue at a first location with a first soft suture anchor and into the first tissue at a second location with a hard-body anchor, wherein the second location is different from the first location, and wherein the flexible coupler is configured to form a sewing pattern over a second tissue to be attached to the first tissue.

19. The surgical construct of claim 18, wherein the sewing pattern is formed by knotlessly connecting the flexible coupler to a plurality of soft anchors, wherein each of the plurality of soft anchors is secured within the first tissue, and wherein the flexible coupler is serially connected to each of the plurality of soft anchors to form the sewing pattern over the second tissue.

20. The surgical construct of claim 18, wherein the first tissue is bone and the second tissue is soft tissue.

21. A method of tissue repair comprising:
securing a plurality of soft anchors into a first tissue;
securing a fixation device to the first tissue; and
knotlessly connecting the fixation device to each of the plurality of soft anchors by a flexible coupler, wherein the flexible coupler is fixedly attached to the fixation device and passes through each of the plurality of soft anchors to form a stitching pattern over a second tissue to be attached to the first tissue.

22. The method of claim 21, further comprising:
passing the flexible coupler through a tubular sheath of a first of the plurality of soft anchors;
subsequently, passing the flexible coupler through a tubular sheath of a second of the plurality of soft anchors;
subsequently, passing the flexible coupler through a tubular sheath of a third of the plurality of soft anchors; and
securing the flexible coupler to the fixation device.

23. The method of claim 22, wherein the fixation device is a hard-body anchor.

24. The method of claim 22, wherein each of the first, second and third of the plurality of soft anchors is an all-suture anchor formed of suture, and wherein the flexible coupler is elastic suture.

25. The method of claim 21, wherein the plurality of soft anchors are secured into the first tissue in a predefined pattern.

26. The method of claim 21, wherein connecting the fixation device to each of the plurality of soft anchors by the flexible coupler is conducted in a sequential manner.

27. The method of claim 21, wherein the first tissue is bone and the second tissue is soft tissue, ligament, or graft.
